(19) United States
(12) Patent Application Publication
(10) Pub. No.: US 2007/0077261 A1
(43) Pub. Date: Apr. 5, 2007

(54) Compositions and method for enhancing the solubility of ascorbic acid using solubilization enhancers

(76) Inventor: Jerry Zhang, Grayslake, IL (US)

Correspondence Address: Jerry Zhang, 1061 Chadwick Dr., Grayslake, IL, 60030, US

(21) Appl. No.: 11/242,306

(22) Filed: Oct. 3, 2005

(51) Int. Cl. A61K 31/375; A61K 9/00

(52) U.S. Cl. 424/400; 514/474

(57) ABSTRACT

A method for dissolving ascorbic acid in a nonaqueous alcohol solvent using a solubilization enhancer is described. The amount of ascorbic acid solubilized in the nonaqueous alcohol solvent containing the solubilization enhancer is greater than the amount of ascorbic acid that would be soluble in the nonaqueous alcohol solvent without the solubilization enhancer. A stable ascorbic acid composition comprising a mixture of the dissolved ascorbic acid and solubilization enhancer is also disclosed.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

This invention concerns a method for enhancing the solubility of ascorbic acid in a nonaqueous alcohol solvent using solubilization enhancers. A stable topical composition comprises a mixture of the dissolved ascorbic acid and solubilization enhancers in the nonaqueous alcohol solvent.

### BACKGROUND OF THE INVENTION

Ascorbic acid, Vitamin C, is known as being suitable for preventing or treating photo-damage to skin. Ascorbic acid is highly soluble, but extremely unstable, in water and rapidly degrades to bio-inactive products. It is known that ascorbic acid is much more stable in nonaqueous polar organic solvents such as compounds having two or more hydroxyl groups per molecule. Unfortunately, its solubility is limited in such solvents, generally no more than a few percent by weight. Since the beneficial effects of ascorbic acid are known to be dose-dependent, it is therefore highly desirable to prepare stable topical composition comprising ascorbic acid dissolved in a dermatologically acceptable vehicle at a concentration of greater than 10% by weight, more preferably up to 40% by weight.

Numerous patents and publications disclose various ways of stabilizing ascorbic acid, especially for low concentrations of ascorbic acid. For example, Takashima, et al., “Ascorbic Acid Esters and Skin Pigmentation” in Am. Perfiumery and Cosmetics 86, 29 (1971) teaches use of the ascorbyl phosphate, an ester of ascorbic acid, that is stable at alkaline pH solutions. The ascorbyl phosphate can be formulated in various topical preparations. However, the ascorbyl phosphate is less bioactive than ascorbic acid.

Choy et al., “Efficient Transdermal Penetration and Improved Stability of L-Ascorbic Acid Encapsulated in an Inorganic Nanocapsule” in Bull. Korean Chem. Soc. 24, 499 (2003) teaches encapsulation of L-ascorbic acid in an inorganic matrix. The encapsulated ascorbic acid is protected by a layered bio-compatible inorganic material. The encapsulated ascorbic acid is not soluble in solvents and can only be suspended.

U.S. Pat. No. 6,361,783 discloses a method of dissolving ascorbic acid in nonaqueous polar organic solvents at high temperatures. Rapid cooling of the mixture to room temperature yields an ascorbic acid solution. Subsequently, the ascorbic acid solution serves as a disperse phase in an emulsion using a nonaqueous silicone vehicle as a continuous phase. The disclosed maximum solubility of ascorbic acid in dermatologically acceptable solvents such as glycerin or propylene glycol is 17% by weight. It is highly desirable to achieve solubility levels of up to 40% by weight for maximum efficacy.

U.S. Pat. No. 6,146,664 discloses a method of suspending ascorbic acid particulate in anhydrous silicone vehicles. The ascorbic acid particulate compositions are stable to moisture and air oxidation. High levels of particulate ascorbic acid can be suspended in the silicone vehicles (up to 40% by weight).

U.S. Pat. No. 5,308,621 teaches a method of stabilizing ascorbic acid. Fine particulate ascorbic acid is suspended in a dermatologically acceptable carrier such as nonaqueous polar alcohol solvents or petroleum jelly. Up to 45% of particulate ascorbic acid can be suspended in the carrier.

As indicated by these references, although ascorbic acid is important to the skin, it is difficult to formulate stabilized topical preparations (such as dermatological or cosmetic formulations), particularly at the higher concentrations needed for maximum efficacy, where ascorbic acid is dissolved in a dermatologically acceptable vehicle, but not merely suspended.

### SUMMARY OF THE INVENTION

Generally speaking, in accordance with the present invention, a method is provided for enhancing the solubility of ascorbic acid in a nonaqueous alcohol solvent using a solubilization enhancer. The solubilization enhancer is a dermatologically acceptable organic compound.

A stable ascorbic acid topical composition comprises a mixture of the dissolved ascorbic acid and solubilization enhancer in the nonaqueous alcohol solvent. The nonaqueous alcohol solvent is a dermatologically acceptable compound.

Accordingly, it is an object of the invention to provide a method for enhancing the solubility of ascorbic acid in the nonaqueous alcohol solvent using the solubilization enhancer.

Another object of the invention is to formulate a stable ascorbic acid topical composition.

A further object of the invention is to formulate a topical composition comprising a mixture of the dissolved ascorbic acid and solubilization enhancer in the nonaqueous alcohol solvent, whereby the presence of the solubilization enhancer increases the solubility of ascorbic acid in the nonaqueous alcohol solvent when compared to the solubility of ascorbic acid in the absence of the solubilization enhancer.

Still other objects and advantages of the invention will, in part, be obvious and will, in part, be apparent from the following detailed description of the preferred embodiments.

### DETAILED DESCRIPTION OF THE PREFERRED EMBODIMENTS

In the disclosed embodiments, a method for enhancing the solubility of ascorbic acid in the nonaqueous alcohol vehicle using the solubilization enhancer is described. A stable topical composition comprises a mixture of the dissolved ascorbic acid and solubilization enhancer in the nonaqueous alcohol vehicle.

The term ‘ascorbic acid’, when used in accordance with the present invention, means L-ascorbic acid, either synthetic or natural, the bio-available form, and derivatives thereof. The term ‘dissolved’ or ‘dissolving’ means that the ascorbic acid or the solubilization enhancer is essentially solubilized in the nonaqueous alcohol solvent, and that the ascorbic acid or the solubilization enhancer will not exist to any appreciable degree in the particulate or crystalline form. Furthermore, the dissolved ascorbic acid or the solubilization enhancer will not crystallize out of the solution upon storage.

A ‘solubilization enhancer’ is a compound that renders sparingly soluble substances more soluble in a solvent.

A ‘nonaqueous’ composition is one that is substantially water free. While water is not intentionally added to a nonaqueous composition, trace amounts of water (for example, existed in the solvent as an impurity) may still be present. It is desired that the amount of water in the composition be less than about 5% by weight, preferably less than 3% by weight.

It has been unexpectedly discovered that the presence of the solubilization enhancer increases the solubility of ascorbic acid in the nonaqueous alcohol solvent when compared to the solubility of ascorbic acid in the absence of the solubilization enhancer. The exact reasons for the surprising effect of the solubilization enhancement are unknown, but it is believed that the solubilization enhancing effect results from the hydrogen bonding and polar properties of the solubilization enhancers.

Ascorbic acid is a polar organic molecule of the general formula: C.sub.6 H.sub.8 O.sub.6. The multiple hydroxyl groups in the ascorbic acid molecule impart its polar property and hydrogen bonding capability. It is believed that the hydrogen bonds exist among the molecules in the mixture, which include the dissolved ascorbic acid and solubilization enhancer molecules as well as the solvent molecules. Furthermore, the polar environment present in the mixture also provides a favorable solubilizing vehicle for ascorbic acid. The dissolved ascorbic acid delivers the intended effect on the skin while being stable and effective.

The dissolved ascorbic acid may be present in an amount of at least 5% by weight, at least 15% by weight, or even as much as 40% by weight. Preferably dissolved ascorbic acid should be present in an amount of 15 to 35% by weight.

It is known that polar organic compounds are likely to dissolve in polar organic solvents. Alcohols are well known polar organic solvents. Suitable alcohol solvents have the general formula of R(OH).sub.n where n is equal to or greater than 1 and R is generally C.sub.2-8 alkyl or substituted alkyl groups. Examples of suitable alcohol solvents are ethyl alcohol, 1 -propanol, 2-propanol, 1-butanol, and mixtures thereof. Particularly preferred alcohols are ethyl alcohol and 2-propanol.

For purposes of this specification, polyols, also known as polyhydric alcohols, are defmed as organic compounds having at least two hydroxyl groups per molecule. The general formula of suitable polyols are: R(OH).sub.n where n is equal to or greater than 2 and R is generally C.sub.2-10 alkyl or substituted alkyl groups. They are polar molecules capable of forming hydrogen bonds with themselves and/or with other polar molecules capable of forming hydrogen bonds.

Polyols are preferred nonaqueous alcohol solvents. Examples of dermatologically acceptable polyols are glycerin, propylene glycol, 1,2-butanediol, 1,3-butanediol, 1,2-pentanediol, 1,5-pentanediol, 1,2-hexanediol, 1,6-hexanediol, diglycerin, dipropylene glycol, 1,2,3-hexanetriol, 1,2,6-hexanetriol or mixtures of the suitable polyols in any given ratio. Especially preferred polyols are glycerin, propylene glycol, and 1,6-hexanediol.

The polyol vehicles may be present in an amount of 20 to 90% by weight, more preferably 40 to 70% by weight.

Preferably the solubilization enhancers are safe, dermatologically acceptable, polar, hydrogen-bonding, organic compounds or mixtures of such compounds. It has been unexpectedly discovered that urea and/or urea derivatives have the desired solubilization enhancing effect. Urea is a diamide of carbonic acid. Urea has the general formula of H.sub.2 N—C(O)—NH.sub.2. Urea has a significant solubility in the polyols. For example, 1 gram of urea dissolves in 2 ml of glycerin (The Merck Index, 11^{th} Edition, 9781). Urea derivatives are derived from urea by substituting one or more of the hydrogen atoms in the urea molecule with other chemical groups. The urea derivative, as used herein, have the general formula of R.sub.3 R.sub.4 N—C(O)—NR.sub.1 R.sub.2, where R.sub. 1, R.sub.2, R.sub.3, and R.sub.4 are each independently hydrogen or C.sub.2-4 alkyl or substituted alkyl groups.

Examples of suitable urea derivatives are hydroxyalkyl urea and alkyl urea where hydroxyalkyl or alkyl groups having at least 2 carbon atoms. The hydroxyalkyl urea, as used herein, have the general formula of HO—[CH.sub.2].sub.n —N(H)—C(O)—NH.sub.2 where n is from 2 to 4. These compounds are also known as mono-substituted hydroxyalkyl urea. The alkyl urea, as used herein, have the general formula of CH.sub.3—[CH.sub.2].sub.n —N(H)—C(O)—NH.sub.2 where n is from 1 to 3. They are known as mono-substituted alkyl urea.

Examples. of the suitable hydroxyalkyl urea are N-2-hydroxyethyl urea, N-3-hydroxypropyl urea, N-2-hydroxypropyl urea, N-4-hydroxybutyl urea, and mixtures thereof. Examples of the suitable alkyl urea are ethyl urea, 1-propyl urea, 2-propyl urea, 1-butyl urea, and mixtures thereof. Mixtures of urea, the hydroxyalkyl urea, and the alkyl urea in any given ratio are also suitable as the solubilization enhancers. Especially preferred solubilization enhancer is urea.

Urea or the urea derivatives are polar molecules having multiple —N—H groups per molecule. They are capable of forming strong hydrogen bonds. Ascorbic acid is also a polar molecule having multiple hydroxyl groups. It is believed that strong polar interactions as well as hydrogen bonds exist between the dissolved urea and/or urea derivative and the dissolved ascorbic acid in the mixture. Without being bound by a particular theory, it is postulated that the hydrogen bonding and polar interactions are responsible for the surprising effect of solubilization enhancement.

It has been discovered in the present invention that the amount of ascorbic acid solubilized in the nonaqueous mixture containing the dissolved solubilization enhancer is greater than the amount of ascorbic acid that would be soluble in the nonaqueous alcohol solvent in the absence of the solubilization enhancer. Most unexpectedly, concentrations of the dissolved ascorbic acid as high as 28% (by weight) have been achieved in the nonaqueous mixture using urea and/or the urea derivatives as the solubilization enhancers.

The dissolved urea or urea derivatives may be present in an amount of at least 5% by weight, at least 10% by weight, or even as much as 40% by weight. Preferably dissolved solubilization enhancers should be present in an amount of about 15 to 35% by weight.

In accordance with the present invention, the preferred method for dissolving ascorbic acid in the nonaqueous alcohol solvent using the solubilization enhancer is as follows:
heating the alcohol solvent to a temperature of about 40 to about 120.degree. C., preferably to a temperature of about 50 to about 100.degree. C.,
adding the solubilization enhancer and the ascorbic acid together while maintaining the desirable temperature, while stirring until dissolved,
cooling the mixture to room temperature.

An alternative method of preparation, where the solubilization enhancer is dissolved first, is described as follows:
heating the alcohol solvent to a temperature of about 40 to about 120.degree. C., preferably to a temperature of about 50 to about 100.degree. C.,
adding the solubilization enhancer while maintaining the desirable temperature, while stirring until dissolved,
adding the ascorbic acid to the mixture while maintaining the desirable temperature, while stirring until dissolved,
cooling the mixture to room temperature.

Yet another alternative method of preparation is that the solubilization enhancer and the ascorbic acid can be added to the alcohol solvent at room temperature even before heat energy is supplied. Then, the alcohol solvent, the solubilization enhancer, and the ascorbic acid can be heated together to the desirable temperature, for example, a temperature of about 40 to about 120.degree. C., preferably to a temperature of about 50 to about 100.degree. C., while stirring until dissolved.

Therefore, it has been found in the present invention that the solubilization enhancing effect is independent of how the solubilization enhancer is added to the mixture. It is believed that it is the chemical and structural properties of the solubilization enhancer that are responsible for the surprising effect of the solubilization enhancement.

After the solubilization enhancer and the ascorbic acid are dissolved in the nonaqueous alcohol solvent, the mixture is allowed to cool to room temperature. The cooled mixture is a solution in which the solubilization enhancer and the ascorbic acid are essentially dissolved.

The nonaqueous ascorbic acid composition of the present invention can contain conventional amounts of one or more other desirable ingredients: vitamins, coenzymes, skin penetration enhancers, surfactants, emulsifiers, or herbal extracts, and even sunscreens. Examples of suitable vitamins and/or coenzymes are: tocopherols (vitamin E), tocopherol acetate, tocopherol succinate, tocotrienols, retinol, vitamin A, vitamin A palmitate, or coenzyme Q-10. They may be present in an amount of at least 0.01% by weight, 1% by weight, or even as much as 10% by weight.

The nonaqueous composition may also comprise aesthetic agents such as silicone oils to achieve more desirable skin feel and consistency. Examples of suitable silicone oils are dimethicone and cyclomethicone. The composition may also comprise antioxidants. The antioxidants are ingredients that would assist in preventing or delaying spoilage. Examples of suitable anti oxidants are propyl gallate, butylated hydroxytoluene, butylated hydroxyanisole, sodium sulfite, and sodium erythrobate. They may be present in an amount of at least 0.01% by weight, or even as much as 5% by weight.

The following examples are included for purposes of illustrating the technology covered by this disclosure. They are not intended to limit the scope of the claimed invention in any manner. One skilled in the art will understand that there are alternatives to these specific embodiments that are not completely described by these examples.

### EXAMPLE 1 (FOR COMPARISON)

This example is to demonstrate that ascorbic acid has lower solubility in propylene glycol in the absence of a solubilization enhancer.

| Component | Amount (weight percentage) |
|---|---|
| Propylene glycol | 80% |
| L-Ascorbic acid | 20% |

The propylene glycol was heated to 75.degree. C. The ascorbic acid was added to the propylene glycol at 75.degree. C. The mixture was maintained at 75.degree. C., while stirring for a minimum of 5 hours. The ascorbic acid was not soluble in the propylene glycol under the experimental condition.

It was also determined that in a composition comprising, by weight, 17% ascorbic acid and 83% propylene glycol prepared by heating the mixture at 75.degree. C., the ascorbic acid was soluble under this experimental condition. This result is consistent with the solubility data disclosed in U.S. Pat. No. 6,361,783.

### EXAMPLE 2 (FOR COMPARISON)

This example is to demonstrate that a polar polyol is not a solubilization enhancer for ascorbic acid in propylene glycol. Sorbitol is a polar polyol having six hydroxyl groups and has the general formula of C.sub.6 H.sub.12 O.sub.6.

| Component | Amount (weight percentage) |
|---|---|
| Propylene glycol | 58% |
| Sorbitol | 22% |
| L-Ascorbic acid | 20% |

The propylene glycol was heated to 85.degree. C. Sorbitol was added to the propylene glycol at 85.degree. C. The mixture was maintained at 85.degree. C., while stirring until sorbitol was dissolved. Then, the. ascorbic acid was added to the mixture at 85.degree. C., while stirring until the ascorbic acid was dissolved. The mixture was cooled down to room temperature and yielded, a clear viscous solution. After standing 24 hours at room temperature, the mixture started turning cloudy. After 48 hours, large amount of crystals were formed at bottom of the sample container.

### EXAMPLE 3

This example is to demonstrate the solubilization enhancing effect of urea in propylene glycol. The experimental condition was same as described in the Example 1. A nonaqueous composition in accordance with the invention was prepared as follows:

| Component | Amount (weight percentage) |
|---|---|
| Propylene glycol | 50% |
| Urea | 22% |
| L-Ascorbic acid | 28% |

The propylene glycol was heated to 75.degree. C. The urea and ascorbic acid were added together to the propylene glycol while maintaining the temperature at 75.degree. C., while stirring until dissolved. It took less than 4 hours for the ascorbic acid and the urea to dissolve in the propylene glycol. The mixture was allowed to cool to room temperature. The mixture was a clear solution.

### EXAMPLE 4

This example is to demonstrate the solubilization enhancing effect of N-2-Hydroxyethyl urea in propylene glycol. A nonaqueous composition in accordance with the invention was prepared as follows:

| Component | Amount (weight percentage) |
|---|---|
| Propylene glycol | 54% |
| N-2-Hydroxyethyl urea | 22% |
| L-Ascorbic acid | 24% |

The propylene glycol was heated to 85.degree. C. The N-2-hydroxyethyl urea and ascorbic acid were added together to the propylene glycol while maintaining the temperature at 85.degree. C., while stirring until dissolved. The mixture was allowed to cool to room temperature. The mixture was a slightly yellow solution.

### EXAMPLE 5

This example is to demonstrate the solubilization enhancing effect when urea is dissolved first in glycerin. A nonaqueous composition in accordance with the invention was prepared as follows:

| Component | Amount (weight percentage) |
|---|---|
| Glycerin | 55% |
| Urea | 20% |
| L-Ascorbic acid | 25% |

The glycerin was heated to 70.degree. C. The urea was added to the glycerin while maintaining the temperature at 70.degree. C., while stirring until dissolved. Then, ascorbic acid was added to the mixture while maintaining the temperature at 70.degree. C., while stirring until dissolved. The mixture was allowed to cool to room temperature. The mixture was a clear solution.

### EXAMPLE 6

This example is to demonstrate the solubilization enhancing effect when urea and ascorbic acid are added together to propylene glycol at room temperature before heating is started. A nonaqueous composition in accordance with the invention was prepared as follows:

| Component | Amount (weight percentage) |
|---|---|
| Propylene glycol | 55% |
| Urea | 20% |
| L-Ascorbic acid | 25% |

The urea and ascorbic acid were added to the propylene glycol at room temperature. The mixture was then heated together to and maintained at 70.degree. C., while stirring until dissolved. The mixture was allowed to cool to room temperature. The mixture was a clear solution.

This confirms that the solubilization enhancer and the ascorbic acid can be added together to the propylene glycol at room temperature before heating is started.

### EXAMPLE 7

This example is to demonstrate the solubilization enhancing effect in a mixture of the polyols. A nonaqueous composition in accordance with the invention was prepared as follows:

| Component | Amount (weight percentage) |
|---|---|
| Propylene glycol | 35% |
| Glycerin | 20% |
| Urea | 20% |
| L-Ascorbic acid | 25% |

The urea and ascorbic acid were added to the propylene glycol and glycerin mixture at room temperature. The mixture was then heated together to and maintained at 70.degree. C., while stirring until dissolved. The mixture was allowed to cool to room temperature. The mixture was a clear solution.

### EXAMPLE 8

This example is to demonstrate a nonaqueous composition comprising vitamins. A nonaqueous composition in accordance with the invention was prepared as follows:

| Component | Amount (weight percentage) |
|---|---|
| Propylene glycol | 51% |
| Polysorbate-20 | 0.75% |
| Sodium laureth sulfate | 0.75% |
| Urea | 22% |
| L-Ascorbic acid | 25% |
| Vitamin E acetate | 0.5% |

The propylene glycol was heated to 65.degree. C. The polysorbate-20 and sodium laureth sulfate were added to the propylene glycol while maintaining the temperature at 65.degree. C., while stirring until dissolved. Then, the urea and ascorbic acid were added to the mixture while maintaining the temperature at 65.degree. C., while stirring until dissolved. Finally, Vitamin E acetate was added to the mixture while maintaining the temperature at 65.degree. C., while stirring until homogenized. The mixture was allowed to cool to room temperature. The mixture was a somewhat translucent solution.

### EXAMPLE 9

Stability studies were performed on the formula of EXAMPLE 5. The sample was prepared under laboratory conditions and stored in dark bottles. The results of the stability study are shown as follows:

| Time | Temperature | Ascorbic acid remaining |
|---|---|---|
| 13 weeks | 25. degree. C. | >90% |
| 26 weeks | 25. degree. C. | >80% |

The results indicate that the ascorbic acid formulation of the present invention exhibits good stability.

The solution remained clear without cloudiness throughout the test period. No evidence of crystal formation or precipitation was evident at room temperature after 26 weeks.

It will thus be seen that the objects set forth above, among those made apparent from the preceding description, are efficiently attained and, since certain changes may be made in carrying out the above process and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible mixtures of such ingredients wherever the sense permits.

What is claimed is:

1. A method for enhancing the solubility of ascorbic acid in a nonaqueous alcohol solvent having at least two carbon atoms using a solubilization enhancer selected from the group consisting of urea, urea derivatives, and mixtures thereof, comprising:
placing the solubilization enhancer and ascorbic acid in intimate contact with the nonaqueous alcohol solvent,
heating the nonaqueous alcohol solvent containing the solubilization enhancer and the ascorbic acid to a temperature of about 40 to about 120.degree. C. for sufficient time to permit the solubilization enhancer and ascorbic acid to dissolve in the nonaqueous alcohol solvent,
cooling the mixture to room temperature,
whereby the amount of ascorbic acid solubilized in the cooled mixture containing the dissolved solubilization enhancer is greater than the amount of ascorbic acid that would be soluble in the nonaqueous alcohol solvent in the absence of the solubilization enhancer.

2. The method of claim 1 wherein the urea derivatives comprise hydroxyalkyl urea.

3. The method of claim 2 wherein the hydroxyalkyl urea is N-2-hydroxyethyl urea.

4. The method of claim 1 wherein the urea derivatives comprise alkyl urea.

5. The method of claim 4 wherein the alkyl urea is ethyl urea.

6. The method of claim 1 wherein the nonaqueous alcohol solvent comprises polyol.

7. The method of claim 6 wherein the polyol is selected from the group consisting of glycerin, propylene glycol, 1,2-butanediol, 1,3-butanediol, 1,2-pentanediol, 1,5-pentanediol, 1,2-hexanediol, 1,6-hexanediol, diglycerin, dipropylene glycol, 1,2,3-hexanetriol, 1,2,6-hexanetriol, and mixtures thereof.

8. A topical composition, comprising, by weight of the total composition:
ascorbic acid, in an amount of about 5 to about 40%,
a solubilization enhancer selected from the group consisting of urea, urea derivatives, and mixtures thereof, in an amount of about 5 to about 40%,
a nonaqueous alcohol solvent, in an amount of about 20 to about 90%,
whereby the presence of the solubilization enhancer increases the solubility of ascorbic acid in the nonaqueous alcohol solvent when compared to the solubility of ascorbic acid in the absence of the solubilization enhancer.

9. The composition of claim 8 wherein the nonaqueous alcohol solvent comprises polyol.

10. The composition of claim 9 wherein the polyol is selected from the group consisting of glycerin, propylene glycol, 1,2-butanediol, 1,3-butanediol, 1,2-pentanediol, 1,5-pentanediol, 1,2-hexanediol, 1,6-hexanediol, diglycerin, dipropylene glycol, 1,2,3-hexanetriol, 1,2,6-hexanetriol, and mixtures thereof.

11. The composition of claim 8 wherein the urea derivatives comprise hydroxyalkyl urea.

12. The composition of claim 11 wherein the hydroxyalkyl urea is N-2-hydroxyethyl urea.

13. The composition of claim 8 wherein the urea derivatives comprise alkyl urea.

14. The composition of claim 8 wherein the solubilization enhancer is urea and the nonaqueous alcohol solvent is selected from the group consisting of glycerin, propylene glycol, 1,6-hexanediol, and mixtures thereof.

15. The composition of claim 8 wherein the solubilization enhancer and ascorbic acid are essentially dissolved in the nonaqueous alcohol solvent.

16. The composition of claim 8 further comprising one or more ingredient selected from the group consisting of vitamin A and derivatives thereof, vitamin E and derivatives thereof, coenzyme Q-10, and mixtures thereof.

17. A composition comprising, by weight of the total composition:
a nonaqueous alcohol solvent, in an amount of about 40 to about 70%,
a solubilization enhancer selected from the group consisting of urea, urea derivatives, and mixtures thereof, in an amount of about 10 to about 30%, ascorbic acid, in an amount of about 10 to about 30%,
whereby the presence of the solubilization enhancer increases the solubility of ascorbic acid in the nonaqueous alcohol solvent when compared to the solubility of ascorbic acid in the absence of the solubilization enhancer.

18. The composition of claim 17 wherein the solubilization enhancer is urea and the nonaqueous alcohol solvent is selected from the group consisting of glycerin, propylene glycol, 1,6-hexanediol, and mixtures thereof.

19. The composition of claim 17 wherein the urea derivatives comprise hydroxyalkyl urea.

20. The composition of claim 17 wherein the solubilization enhancer and ascorbic acid are essentially dissolved in the nonaqueous alcohol solvent.
